(19) United States
(12) Patent Application Publication
(10) Pub. No.: US 2022/0370049 A1
(43) Pub. Date: Nov. 24, 2022

(54) ORAL ASSEMBLY

(71) Applicant: SHINY TOOTHBRUSH, INC., Incline Village, NV (US)

(72) Inventors: Murat Gocmen, Walnut Creek, CA (US); Ulas Tigin, Istanbul (TR)

(21) Appl. No.: 17/751,278

(22) Filed: May 23, 2022

Related U.S. Application Data
(60) Provisional application No. 63/192,525, filed on May 24, 2021.

(51) Int. Cl. A61B 10/00; A61C 15/04; A61C 19/06

(52) U.S. Cl. CPC A61B 10/0051; A61C 15/046; A61C 19/063

(57) ABSTRACT

An oral assembly (100) for use within a mouth of a user. The oral assembly (100) includes an oral device (104) and a collection device (224). The oral device (104) is configured to be usable within the mouth of the user to provide oral health care treatment. The collection device (224) is coupled to the oral device (104). The collection device (224) is movable between (i) a collection mode wherein the collection device (224) collects saliva from the mouth of the user, and (ii) an expulsion mode wherein the collection device (224) expels a liquid into the mouth of the user. A collection tube (226) can be coupled to the collection device (224). The collection tube (226) can be configured to receive saliva that is collected by the collection device (224) from the mouth of the user.

### RELATED APPLICATION

This application claims priority on U.S. Provisional Application Ser. No. 63/192,525, filed on May 24, 2021, and entitled “ORAL ASSEMBLY.” As far as permitted, the contents of U.S. Provisional Application Ser. No. 63/192,525 are incorporated in their entirety herein by reference.

### BACKGROUND

Individuals often lack the skills and tools to test and monitor their health on their own, especially at home or without the involvement of a lab or medical professional. People want to control their own lives and understand what's happening to their bodies, but currently can do very little along these lines without medical and/or professional supervision.

### SUMMARY

The present invention is directed toward an oral assembly for use within a mouth of a user. In various embodiments, the oral assembly includes an oral device and a collection device. The oral device is configured to be usable within the mouth of the user to provide oral health care treatment. The collection device is coupled to the oral device. The collection device is movable between (i) a collection mode wherein the collection device collects saliva from the mouth of the user, and (ii) an expulsion mode wherein the collection device expels a liquid into the mouth of the user.

In some embodiments, in the expulsion mode, the collection device cooperates with the oral device to floss teeth within the mouth of the user with the liquid.

In certain embodiments, the oral assembly can further include a motor that is coupled to the collection device, the motor being configured to move the collection device between the collection mode and the expulsion mode.

In various embodiments, the oral assembly can further include an assembly housing that houses the collection device.

In some embodiments, the oral assembly can further include a housing cap that is removably attachable to the assembly housing, the assembly housing having an assembly housing axis. The housing cap can be configured to be rotatable about the assembly housing axis.

In certain embodiments, the assembly housing includes a first attachment aperture that is configured to receive a first attachment, the first attachment aperture being configured to activate the first attachment upon insertion.

In various embodiments, the assembly housing includes a second attachment aperture that is configured to receive a second attachment, the second attachment aperture being configured to activate the second attachment upon insertion.

In some embodiments, the housing cap includes a housing cap aperture, and rotation of the housing cap exposes the first attachment aperture and the second attachment aperture when they are aligned with the housing cap aperture.

In certain embodiments, the housing cap is selectively lockable when at least one of the first attachment aperture and the second attachment aperture is exposed.

In various embodiments, the oral assembly can further include a collection tube that is coupled to the collection device, the collection tube being configured to receive saliva that is collected by the collection device from the mouth of the user.

In some embodiments, the oral assembly can further include a saliva storer that is configured to store the saliva collected by the collection device from the mouth of the user.

The present invention is further directed toward an oral assembly for use within a mouth of a user. In various embodiments, the oral assembly includes an oral device, a collection device, a collection tube, and a saliva storer. The oral device is configured to be usable within the mouth of the user to provide oral health care treatment. The collection device is coupled to the oral device. The collection device is movable between (i) a collection mode wherein the collection device collects saliva from the mouth of the user, and (ii) an expulsion mode wherein the collection device expels a liquid into the mouth of the user. The collection tube is coupled to the collection device, the collection tube being configured to receive saliva that is collected by the collection device from the mouth of the user. The saliva storer is configured to store the saliva collected by the collection device from the mouth of the user.

In certain embodiments, the oral assembly further includes an extension tube that selectively connects to at least one of the collection tube and the saliva storer.

In various embodiments, the extension tube includes an extension tube cap that threadedly engages the extension tube to secure the contents of the saliva storer.

In some embodiments, the saliva storer is selectively detachable from the collection device.

In certain embodiments, the saliva storer includes a storer disc having a storer disc tube that is selectively sealable using a sealing cap.

In various embodiments, the storer disc is configured to be removable from the saliva storer.

In some embodiments, the oral assembly further includes a device cover that is configured to (i) cover the collection device and (ii) selectively attach to the oral assembly.

In certain embodiments, in the expulsion mode, the collection device cooperates with the oral device to floss teeth within the mouth of the user with the liquid.

The present invention is also directed toward an oral assembly for use within a mouth of a user. In various embodiments, the oral assembly can include an oral device, a collection device, a collection tube, a motor, and an assembly housing. The oral device is configured to be usable within the mouth of the user to provide oral health care treatment. The collection device is coupled to the oral device. The collection device is movable between (i) a collection mode wherein the collection device collects saliva from the mouth of the user, and (ii) an expulsion mode wherein the collection device expels a liquid into the mouth of the user. The collection tube can be coupled to the collection device. The collection tube can be configured to receive saliva that is collected by the collection device from the mouth of the user. A motor can be coupled to the collection device. The motor can be configured to move the collection device between the collection mode and the expulsion mode.

The assembly housing can house the collection device. The assembly housing can have an assembly housing axis. The assembly housing can include a first attachment aperture that is configured to receive a first attachment. The first attachment can be activated by inserting the first attachment into the first attachment aperture. The assembly housing can include a second attachment aperture that is configured to receive a second attachment. The second attachment can be activated by inserting the second attachment into the second attachment aperture.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a front view of one embodiment of an oral assembly having features of the present invention;

FIG. 2 is a simplified perspective partially exploded view of one embodiment of a portion of the oral assembly;

FIG. 3 is a simplified perspective partially transparent view of one embodiment of a portion of the oral assembly;

FIG. 4A is a perspective view of a portion of the oral assembly, including one embodiment of a collection device;

FIG. 4B is a perspective view of a portion of the oral assembly, including one embodiment of a collection device;

FIG. 5 is a side view of another embodiment of the oral assembly;

FIG. 6 is a perspective view of a portion of the oral assembly, including one embodiment of an oral device;

FIG. 7 is a perspective view of another embodiment of the oral assembly;

FIG. 8 is a perspective view of another embodiment of the oral assembly;

FIG. 9 is a top perspective view of one embodiment of a portion of the oral assembly; and

FIG. 10 is a perspective view of one embodiment of an oral system, including a plurality of oral assemblies and a charger.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of fully inclusive oral assembly for the maintenance of oral health and the analysis of health and wellness via saliva samples. In particular, the oral assembly can expel water into the user's mouth for cleaning purposes. The oral assembly can be reversible so that the oral assembly can collect saliva samples from the user's mouth. The saliva can be extracted from the oral assembly and shipped for analysis. The oral assembly can enable the user to monitor their health and ongoing wellness without having to visit a health practitioner or clinic.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

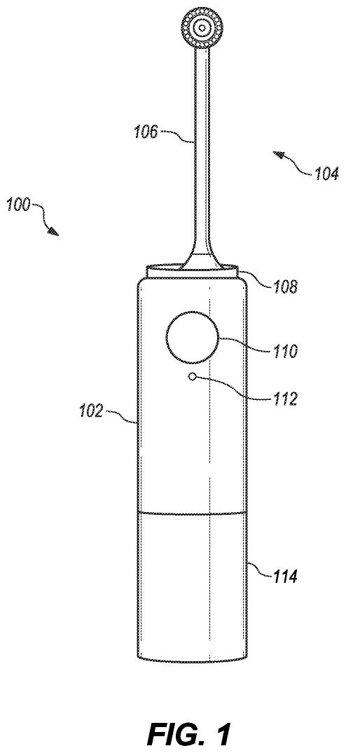

FIG. 1 is a front view of one embodiment of an oral assembly 100. The oral assembly 100 can be used within a user's mouth in order to provide complete oral health care for the user. The oral assembly 100 can also be used within an animal's mouth in order to provide complete oral health care for the animal. Oral health care functionality of the oral assembly 100 can include teeth brushing, teeth flossing, gum, and teeth water treatment. In some embodiments, attachments can be added to the oral assembly 100 to allow for teeth whitening and other aesthetic treatments. The oral assembly 100 can also be used to collect saliva samples for analysis.

The oral assembly 100 can vary depending on the design requirements of the oral assembly 100. It is understood that the oral assembly 100 can include additional components, systems, subsystems, and elements other than those specifically shown and/or described herein. Additionally, or alternatively, the oral assembly 100 can omit one or more of the components, systems, subsystems, and elements that are specifically shown and/or described herein. In some embodiments, various components of the oral assembly 100 can be positioned in a different manner than what is specifically illustrated in FIG. 1.

As illustrated in the embodiment depicted in FIG. 1, the oral assembly 100 can include an assembly housing 102 and an oral device 104. While one oral device 104 is depicted in FIG. 1, it is understood that the oral assembly 100 can include any suitable number of oral devices 104 and/or attachments.

The assembly housing 102 houses and/or protects the interior components of the oral assembly 100. The assembly housing 102 can vary depending on the design requirements of the oral assembly 100. It is understood that the assembly housing 102 can include additional components, systems, subsystems, and elements other than those specifically shown and/or described herein. Additionally, or alternatively, the assembly housing 102 can omit one or more of the components, systems, subsystems, and elements that are specifically shown and/or described herein. In some embodiments, various components of the oral assembly housing 102 can be positioned in a different manner than what is specifically illustrated in FIG. 1.

The assembly housing 102 can include one or more apertures and/or attachment points (to be described in further detail herein). The oral device 104 can be coupled, selectively secured, and/or attached to the assembly housing 102 via the apertures and/or attachment points.

The assembly housing 102 can include any number of suitable devices. The devices can be, or their functionality can include, integrated circuits, processors, controllers, microprocessors, diodes, capacitors, power storage elements, antennae, ASICs, sensors, image elements (e.g., CMOS, CCD imaging elements), amplifiers, ND, and D/A converters, associated differential amplifiers, buffers, microprocessors, optical collectors, transducer including electro-mechanical transducers, piezoelectric actuators, light-emitting electronics which include LEDs, logic, memory, clock, and transistors including active matrix switching transistors, and combinations thereof. The purpose and advantage of using standard ICs (in embodiments, CMOS, on single crystal silicon) are to have and use high quality, high performance, and high functioning circuit components that are also already commonly mass-produced with well-known processes, and which provide a range of functionality and generation of data far superior to that produced by a passive means.

Components within electronic devices or devices are described herein and include those components described herein. A component can be one or more of any of the electronic devices described herein and/or may include a photodiode, LED, TUFT, electrode, semiconductor, other light-collecting/detecting components, transistor, contact pad capable of contacting a device component, thin-film devices, circuit elements, control elements, microprocessors, interconnects, contact pads, capacitors, resistors, inductors, a memory element, power storage element, antenna, logic element, buffer and/or other passive or active components. A device component may be connected to one or more contact pads as known in the art, such as metal evaporation, wire bonding, application of solids or conductive pastes, and the like.

The oral device 104 is used within the user's mouth to provide oral health care treatment. The oral device 104 can be any number of dental, orthodontic, aesthetic, and/or therapeutic devices that can be implemented on the oral assembly 100. The oral device 104 can be a toothbrush, a flosser, a water expulsion device, and a teeth whitener, as non-limiting, non-exclusive examples.

The oral device 104 can vary depending on the design requirements of the oral assembly 100 and/or the assembly housing 102. It is understood that the oral device 104 can include additional components, systems, subsystems, and elements other than those specifically shown and/or described herein. Additionally, or alternatively, the oral device 104 can omit one or more of the components, systems, subsystems, and elements that are specifically shown and/or described herein. In some embodiments, various components of the oral device 104 can be positioned in a different manner than what is specifically illustrated in FIG. 1. The oral device 104 can include a number of components, such as a first attachment 106. Additionally, in some embodiments (such as FIG. 1), the oral device 104 can be covered and/or protected by an oral device cover 114 that can be fixedly secured to the assembly housing 102. The oral device 104 can be coupled to any portion of the oral assembly 100.

As used herein, “coupled” can be understood to mean, without limitation, one or more of the following: attached (selectively and/or removably), bolted, constrained to, engaged (mutually and/or unilaterally), fixed, integrally formed with, interlocked, locked, mated, mounted (pivotally and/or rotatably), secured, set, joined, linked, welded or otherwise connected. It is appreciated that the coupling can be permanent or temporary.

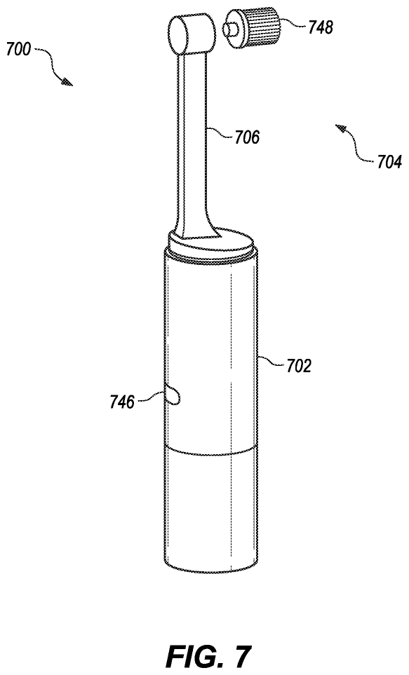

The first attachment 106 can be a toothbrush (as shown in the embodiment illustrated in FIG. 1), including a detachable toothbrush head (shown in greater detail in FIG. 7). As shown in FIG. 1, the first attachment 106 can attach to the assembly housing 102 via a housing cap 108. The first attachment 106 can vary depending on the design requirements of the oral assembly 100 and/or the assembly housing 102. It is understood that the first attachment 106 can include additional components, systems, subsystems, and elements other than those specifically shown and/or described herein. Additionally, or alternatively, the first attachment 106 can omit one or more of the components, systems, subsystems, and elements that are specifically shown and/or described herein. In some embodiments, it is understood that various components of the first attachment 106 can be positioned in a different manner than what is specifically illustrated in FIG. 1.

The housing cap 108 can be coupled to the assembly housing 102 and/or threadedly engage the assembly housing 102 at any suitable location. In the embodiment illustrated in FIG. 1, the housing cap 108 is shown in a fastened state, at the top of the assembly housing 102. The housing cap 108 can secure and/or engage any portion of the assembly housing 102. The housing cap 108 can create an air-tight and liquid-tight seal so that no liquids or gases escape the assembly housing 102 during use and operation of the oral assembly 100. The housing cap 108 can be configured to receive a number of components, such as the first attachment 106. Other oral devices 104 can also be engaged to the housing cap 108.

In certain embodiments, when an attachment or component (such as the first attachment 106) is attached and/or secured to the housing cap 108, the attached attachment or component can be powered on or operationalized. In other words, in some embodiments, if the first attachment 106 is secured to the housing cap 108, the first attachment 106 is activated by the housing cap 108 to become operational. The housing cap 108 can include the necessary components and electronics in order to operationalize the first attachment 106.

The housing cap 108 can vary depending on the design requirements of the oral assembly 100, the assembly housing 102, the oral device 104, and/or first attachment 106. It is understood that the housing cap 108 can include additional components, systems, subsystems, and elements other than those specifically shown and/or described herein. Additionally, or alternatively, the housing cap 108 can omit one or more of the components, systems, subsystems, and elements that are specifically shown and/or described herein. In some embodiments, it is recognized that various components of the housing cap 108 can be positioned in a different manner than what is specifically illustrated in FIG. 1.

As illustrated in the embodiment shown in FIG. 1, the assembly housing 102 can further include a power button 110 and a power indicator 112. The power button 110 can be engageable by the user in order to toggle systems, devices, and/or components of the oral assembly 100. In some embodiments, the power button 110 can be used to engage and disengage the operation of the oral device 104 (such as the first attachment 106). The power button 110 can also be used as a switch that switches the direction of liquids flowing through the oral assembly.

The power indicator 112 can indicate the status of the operation of the attachments, components, and/or devices of the oral assembly 100. In some embodiments, the power indicator 112 can emit light to indicate that the oral device 104 is currently operating. When the user engages the power button 110 to turn off the oral device 104, the power indicator 112 can cease emission of the light to indicate that the oral device 104 is no longer in operation. The power indicator 112 can also be used as an indicator that indicates the direction of liquids flowing through the oral assembly 100 (e.g., in a collection mode, an expulsion mode, or both, as described in further detail herein).

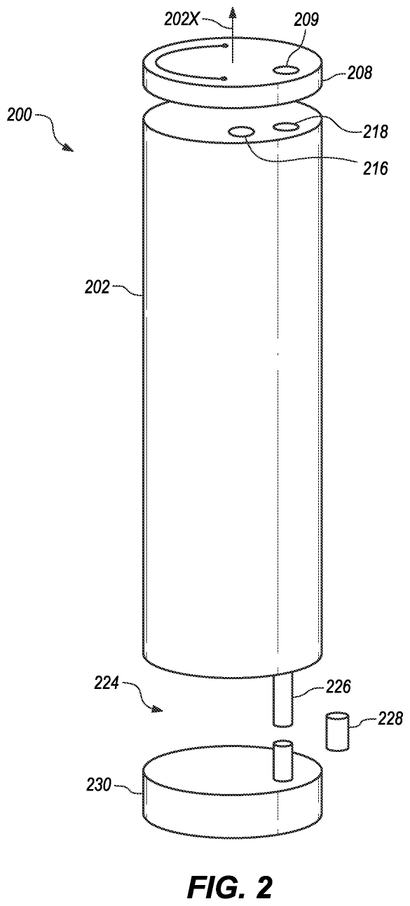

FIG. 2 is a simplified perspective partially exploded view of one embodiment of a portion of the oral assembly 200. As shown in the embodiment illustrated in FIG. 2, the assembly housing 202 can have an assembly housing axis 202X. In certain embodiments, the housing cap 208 can be coupled and/or threadedly secured to the assembly housing 202 by rotating the housing cap 208 about the assembly housing axis 202X. The housing cap 208 can have a housing cap aperture 209 that rotates in unison with the housing cap 208 about the assembly housing axis 202X. In some embodiments, the oral assembly 200 can include a collection device 224.

The housing cap aperture 209 rotates about the assembly housing axis 202X to reveal a first attachment aperture 216 and/or a second attachment aperture 218. The housing cap aperture 209 can also be rotated to hide the first attachment aperture 216 and/or the second attachment aperture 218 to protect the interior portions of the assembly housing 202.

The housing cap aperture 209 can vary depending on the design requirements of the oral assembly 200, the assembly housing 202, the first attachment aperture 216, and/or the second attachment aperture 218. It is understood that the housing cap aperture 209 can include additional components, systems, subsystems, and elements other than those specifically shown and/or described herein. Additionally, or alternatively, the housing cap aperture 209 can omit one or more of the components, systems, subsystems, and elements that are specifically shown and/or described herein. In some embodiments, the housing cap aperture 209 can be positioned in a different manner than what is specifically illustrated in FIG. 2. In other embodiments, the housing cap 208 can include a plurality of housing cap apertures 209 substantially similar to the housing cap aperture 209 described herein.

The first attachment aperture 216 can be configured to receive the first attachment 106 and/or a second attachment (not shown in FIG. 2). The first attachment aperture 216 can power and/or operationalize the first attachment 106 and/or a second attachment upon insertion into the first attachment aperture 216. The first attachment aperture 216 can include the necessary components and electronics in order to operationalize the first attachment 106 and/or the second attachment.

The first attachment aperture 216 can vary depending on the design requirements of the oral assembly 200, the assembly housing 202, and/or the first attachment 106. It is understood that the housing cap aperture 209 can include additional components, systems, subsystems, and elements other than those specifically shown and/or described herein. Additionally, or alternatively, the first attachment aperture 216 can omit one or more of the components, systems, subsystems, and elements that are specifically shown and/or described herein. In some embodiments, the first attachment aperture 216 can be positioned in a different manner than what is specifically illustrated in FIG. 2. As used herein, the first attachment aperture 216 and second attachment aperture 218 are merely demonstrative, and it is understood that the assembly housing 202 could include any number of attachment apertures to receive any number of attachments and/or oral devices 104.

The second attachment aperture 218 can be configured to receive the first attachment 106 and/or the second attachment (not shown in FIG. 2). In some embodiments, the second attachment aperture 218 can be substantially similar in design and/or function to the first attachment aperture 216.

The collection device 224 can collect and/or suction liquids such as saliva from the user. Portions of the collection device 224 can be removed so the saliva can be sent for analysis. The collection device 224 can work in cooperation with the oral device 204, the first attachment 106, and/or any other suitable attachment to collect and/or suction liquids. The collection device 224 can be movable between (i) a collection mode wherein the collection device 224 collects saliva from the mouth of the user, and (ii) an expulsion mode wherein the collection device 224 expels a liquid into the mouth of the user.

For example, in some embodiments, the user can brush their teeth using the first attachment 106 and upon completion of the oral cleaning, engage the collection device 224 to collect the human saliva through the first attachment 106.

In other embodiments, the collection device 224 can be a water expulsion device that is reversible. The water expulsion device can expel water to clean the user's gums and/or teeth and upon completion of the oral cleaning, reverse the direction of the collection device 224 to a suction mode in order to collect the human saliva through the collection device 224.

The collection device 224 can vary depending on the design requirements of the oral assembly 200, the assembly housing 202, the oral device 204, the first attachment 106, and/or any suitable attachment. It is understood that the collection device 224 can include additional components, systems, subsystems, and elements other than those specifically shown and/or described herein. Additionally, or alternatively, the collection device 224 can omit one or more of the components, systems, subsystems, and elements that are specifically shown and/or described herein. In some embodiments, the collection device 224 can be positioned in a different manner than what is specifically illustrated in FIG. 2. It is understood that the oral assembly 200 could include any number of collection devices 224. In various embodiments, the collection device 224 can include a collection tube 226, an extension tube 228, and a saliva storer 230.

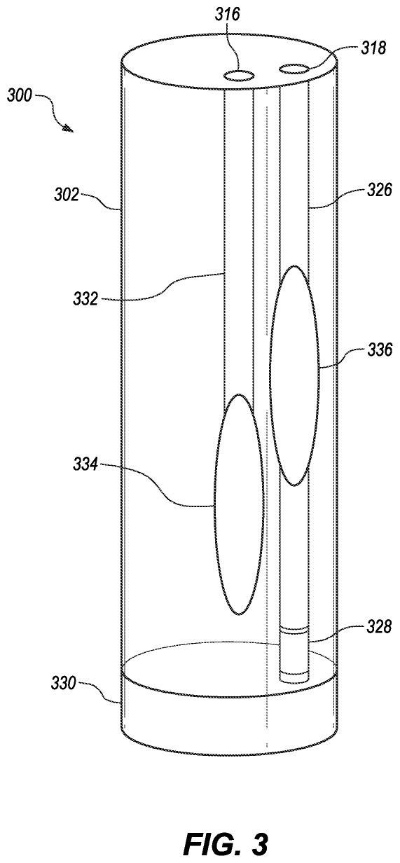

The collection tube 226 is configured to guide liquids in certain directions. For example, in some embodiments, the collection tube 226 can guide water towards the oral device 104 (not shown in FIG. 2) so that the first oral device 104 can expel water into the mouth of the user. The collection tube 226 can also guide water in the opposite direction, toward the saliva storer 230. The collection tube 226 can work in cooperation with any number of motors (shown in FIG. 3 and described herein) and/or pumps to drive the flow direction of the liquids through the collection tube 226.

The collection tube 226 can vary depending on the design requirements of the oral assembly 200, the assembly housing 202, the oral device 204, and/or the collection device 224. It is understood that the collection tube 226 can include additional components, systems, subsystems, and elements other than those specifically shown and/or described herein. Additionally, or alternatively, the collection tube 226 can omit one or more of the components, systems, subsystems, and elements that are specifically shown and/or described herein. In some embodiments, the collection tube 226 can be positioned in a different manner than what is specifically illustrated in FIG. 2. It is understood that the collection device 224 could include any number of collection tubes 226.

The collection tube 226 can extend through the interior of the assembly housing 202 and can be connected to other components of the oral assembly 200, such as motors and/or pumps. The collection tube 226 can include threading and/or a male portion that engages the extension tube 228.

The extension tube 228 can interconnect the collection tube 226 to a tubular portion of the saliva storer 230. The extension tube 228 can include one or more female portions that receive male portions of both the saliva storer 230 and the collection tube 226. The extension tube 228 can vary depending on the design requirements of the oral assembly 200, the assembly housing 202, the oral device 204, the collection device 224, and/or the saliva storer 230. In some embodiments, the extension tube 228 can be positioned in a different manner than what is specifically illustrated in FIG. 2. It is understood that the collection device 224 could include any number of extension tubes 228.

The saliva storer 230 can store the human saliva sample collected via the collection device 224 and/or the oral device 104. The saliva storer 230 can be interconnected to the collection device 224 via the collection tube 226 and/or the extension tube 228. The saliva storer 230 can vary depending on the design requirements of the oral assembly 200, the assembly housing 202, the oral device 204, the collection device 224, and/or the Extension tube 228. In some embodiments, the saliva storer 230 can be positioned in a different manner than what is specifically illustrated in FIG. 2. It is understood that the saliva storer 230 could include any number of tubular portions and/or components that interconnect with the collection tube 226 and/or the extension tube 228.

FIG. 3 is a simplified perspective partially transparent view of one embodiment of a portion of the oral assembly 300. As illustrated in the embodiment displayed in FIG. 3, the assembly housing 302 can include one or more interior components. The assembly housing 302 can further include an oral device tube 332, a first motor 334, and a second motor 336. It is appreciated that the oral assembly 300 can include any number of tubes and/or motors.

The oral device tube 332 can be fixedly secured to the first attachment aperture 316 and the first motor 334. The oral device tube 332 can receive a portion of the oral device 104, first attachment 106, and/or second attachment (not shown in FIG. 3). The oral device tube 332 can direct the flow of liquids from the first motor 334 towards the first attachment aperture 316 so that the liquids can be utilized by the oral device 104, first attachment 106, and/or second attachment. In some embodiments, the oral device tube 332 can be interconnected to a liquid expulsion device and/or a toothbrush.

The oral device tube 332 can vary depending on the design requirements of the oral assembly 300, the assembly housing 302, the oral device 204, the collection device 224, and/or the extension tube 328. In some embodiments, the oral device tube 332 is positioned in a different manner than what is specifically illustrated in FIG. 3. It is understood that the oral device tube 332 can include any number of tubular portions and/or components that interconnect with the first attachment aperture 316 and/or the first motor 334.

The first motor 334 can drive the flow of liquids through the oral device 104. In some embodiments, the first motor 334 drives the flow of liquids through the oral device tube 332 towards the first attachment aperture 316 so that the liquids can be utilized by the oral device 104, first attachment 106, and/or second attachment. The motors 334, 336 can be configured to move the collection device 224 (illustrated in FIG. 2) between the collection mode and/or the expulsion mode.

The first motor 334 can vary depending on the design requirements of the oral assembly 300, the assembly housing 302, the oral device 204, and/or the oral device tube 332. It is understood that the first motor 334 can include additional components, systems, subsystems, and elements other than those specifically shown and/or described herein. Additionally, or alternatively, the first motor 334 can omit one or more of the components, systems, subsystems, and elements that are specifically shown and/or described herein. In some embodiments, the first motor 334 is positioned in a different manner than what is specifically illustrated in FIG. 3.

The second motor 336 can drive the flow of liquids through the collection device 224. In some embodiments, the second motor 336 drives the flow of liquids through the collection tube 326 towards the second attachment aperture 318 so that the liquids can be utilized by the oral device 104, first attachment 106, and/or second attachment. The second motor 336 can be reversible so that it drives the flow of liquids from the oral device 104, first attachment 106, and/or second attachment through the second motor 336 toward the extension tube 328 and/or the saliva storer 330.

The second motor 336 can vary depending on the design requirements of the oral assembly 300, the assembly housing 302, the collection device 224, the collection tube 326, the extension tube 328, and/or the saliva storer 330. It is understood that the second motor 336 can include additional components, systems, subsystems, and elements other than those specifically shown and/or described herein. Additionally, or alternatively, the second motor 336 can omit one or more of the components, systems, subsystems, and elements that are specifically shown and/or described herein. In some embodiments, the second motor 336 is positioned in a different manner than what is specifically illustrated in FIG. 3.

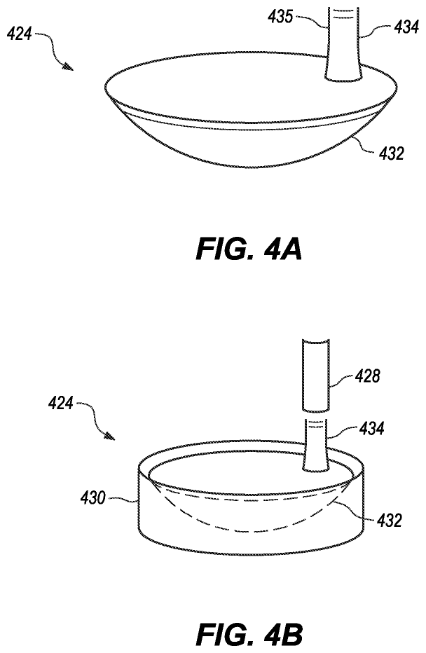

FIG. 4A is a perspective view of a portion of the oral assembly 100, including one embodiment of a collection device 424. As illustrated in the embodiment displayed in FIG. 4A, the collection device 424 can include a storer disc 432, a storage disc tube 434, and a sealing cap 435.

The storer disc 432 can store the saliva sample for analysis. The storer disc 432 can vary depending on the design requirements of the oral assembly 300, the assembly housing 302, the collection device 424, the collection tube 326, the extension tube 428, and/or the saliva storer 430. It is understood that the storer disc 432 can include additional components, systems, subsystems, and elements other than those specifically shown and/or described herein. Additionally, or alternatively, the storer disc 432 can omit one or more of the components, systems, subsystems, and elements that are specifically shown and/or described herein.

The storage disc tube 434 can be integrally formed with the storer disc 432. The storage disc tube 434 can vary depending on the design requirements of the oral assembly 300, the assembly housing 302, the oral device 204, the collection device 424, and/or the extension tube 428. In some embodiments, the storage disc tube 434 is positioned in a different manner than what is specifically illustrated in FIG. 4A. It is understood that the storage disc tube 434 could include any number of tubular portions and/or components that interconnect with the collection tube 326, the extension tube 428, and/or the sealing cap 435. In some embodiments, the storage disc tube 434 can include a male portion similar to the collection tube 326 so that the storage disc tube 434 can be secured to the female portion of the extension tube 428.

The sealing cap 435 can be sealingly attached and/or coupled to any portion of the collection device 424 in order to seal the collection device 424 and prevent any of the saliva sample(s) from exiting the collection device 424. As shown in the embodiment illustrated in FIG. 4A, the sealing cap 435 can be threadedly secured and/or coupled to the storage disc tube 434. The sealing cap 435 can vary depending on the design requirements of the oral assembly 300, the assembly housing 302, the oral device 204, the collection device 424, the extension tube 428, and/or the storage disc tube 434.

In some embodiments, the sealing cap 435 is positioned in a different manner than what is specifically illustrated in FIG. 4A. It is understood that the sealing cap 435 could include any number of components that interconnect with the storage disc tube 434 and/or any portion of the collection device 424. In some embodiments, the sealing cap 435 can include a female portion similar to the extension tube 428 so that the sealing cap 435 can be secured to the male portion of the storage disc tube 434.

FIG. 4B is a perspective view of a portion of the oral assembly 100, including one embodiment of a collection device 424. As illustrated in the embodiment shown in FIG. 4B, the storer disc 432 is shown in phantom to display how the storer disc 432 can be fitted into the saliva storer 430. The size and/or positioning of the storer disc 432 can vary depending on the design requirements of the saliva storer 430. Also illustrated in the embodiment shown in FIG. 4B, the extension tube 428 is displayed as being detached from the storage disc tube 434. The extension tube 428 and storage disc tube 434 can be interconnected by any suitable methods known in the art.

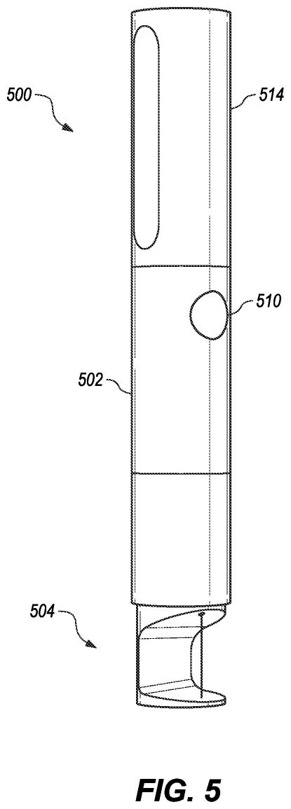

FIG. 5 is a perspective view of another embodiment of the oral assembly 500. As illustrated in the embodiment shown in FIG. 5, the oral assembly 500 can include the assembly housing 502, an oral device 504, the power button 510, and the oral device cover 514.

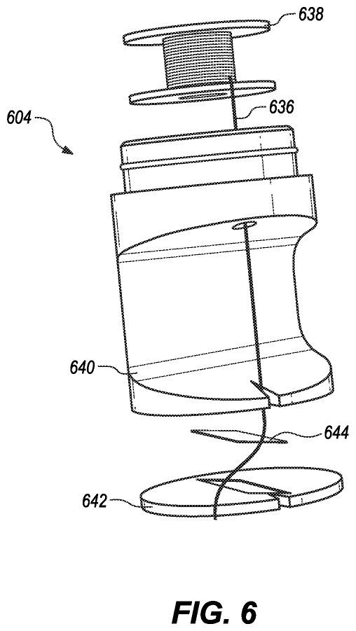

The oral device 504 can be substantially similar to the oral device 104 shown in FIG. 1, or it can be an entirely distinct device. One embodiment of the oral device 504 will be shown in FIG. 6 and described in greater detail herein.

As shown in FIG. 5, the oral device cover 514 can be fixedly attached to the assembly housing 502 in order to cover and/or protect the oral device 504 and/or the first attachment 106. The oral device cover 514 can also be used to cover and/or protect the oral device 504. The oral device 504 can vary depending on the design requirements of the oral assembly 500 and/or the assembly housing 502. It is understood that the oral device 504 can include additional components, systems, subsystems, and elements other than those specifically shown and/or described herein. Additionally, or alternatively, the oral device 504 can omit one or more of the components, systems, subsystems, and elements that are specifically shown and/or described herein. In some embodiments, various components of the oral device 504 can be positioned in a different manner than what is specifically illustrated in FIG. 5.

FIG. 6 is a perspective view of a portion of the oral assembly 100, including one embodiment of an oral device 604. As illustrated in the embodiment shown in FIG. 6, the oral device 604 can be utilized as a dental flossing assembly. The oral device 604 can include dental floss 636, a floss spool 638, a floss assembly arm 640, a floss assembly cap 642, and a floss assembly partition 644.

The dental floss 636 can be utilized by the user in order to floss the user's teeth. The dental floss 636 can be formed using any suitable material to floss the user's teeth, including synthetic and natural materials. The dental floss 636 can vary depending on the design requirements of the oral assembly 100, the oral device 604, the floss spool 638, the floss assembly arm 640, the floss assembly cap 642, and/or the floss assembly partition 644. In some embodiments, the dental floss 636 is positioned in a different manner than what is specifically illustrated in FIG. 6.

The floss spool 638 can be configured to receive and/or store the dental floss 636. In some embodiments, the dental floss 636 can be wound and/or spooled around the floss spool 638 for storage. As the user uses the dental floss 636, the user can unwind the dental floss 636 from the floss spool 638 in order to access the usable portions of the dental floss 636.

The floss assembly arm 640 can retain the portion of the dental floss 636 that the user will be using to floss the user's teeth. The floss assembly arm 640 can be substantially u-shaped in order to accommodate the curvature of the user's teeth and to facilitate flossing between the user's teeth. The user can pull the dental floss 636 through the floss assembly arm 640 after the flossing is complete to retrieve the unused portion of the dental floss 636.

The floss assembly arm 640 can vary depending on the design requirements of the oral assembly 100, the oral device 604, the dental floss 636, the floss spool 638, the floss assembly arm 640, the floss assembly cap 642, and/or the floss assembly partition 644. It is understood that the floss assembly arm 640 can include additional components, systems, subsystems, and elements other than those specifically shown and/or described herein. Additionally, or alternatively, the floss assembly arm 640 can omit one or more of the components, systems, subsystems, and elements that are specifically shown and/or described herein. In some embodiments, various components of the floss assembly arm 640 can be positioned in a different manner than what is specifically illustrated in FIG. 6.

The floss assembly cap 642 can be fixedly secured to the floss assembly arm 640 so that the dental floss 636 can be fixed in place during flossing. Upon completion of the flossing, the floss assembly cap 642 can be detached from the floss assembly arm 640 so that the used portion of the dental floss 636 can be removed and the unused portion of the dental floss 636 can be moved into position within the floss assembly arm 640.

The floss assembly cap 642 can vary depending on the design requirements of the oral assembly 100, the oral device 604, the dental floss 636, the floss spool 638, the floss assembly arm 640, and/or the floss assembly partition 644. It is understood that the floss assembly cap 642 can include additional components, systems, subsystems, and elements other than those specifically shown and/or described herein. Additionally, or alternatively, the floss assembly cap 642 can omit one or more of the components, systems, subsystems, and elements that are specifically shown and/or described herein. In some embodiments, floss assembly cap 642 can be positioned in a different manner than what is specifically illustrated in FIG. 6.

The floss assembly partition 644 retains the positioning of the dental floss 636 between the floss assembly arm 640 and the floss assembly cap 642. The floss assembly partition 644 can be fixedly secured into portions of both the floss assembly arm 640 and the floss assembly cap 642 so that the floss assembly cap 642 can be removably attached to both the floss assembly arm 640 and the floss assembly partition 644 while securing the dental floss 636 in the desired position. The floss assembly partition 644 can also cut the dental floss 636 in order to remove the used portion of the dental floss 636.

The floss assembly partition 644 can vary depending on the design requirements of the oral assembly 100, the oral device 604, the dental floss 636, the floss spool 638, the floss assembly arm 640, and/or the floss assembly cap 642. It is understood that the floss assembly partition 644 can include additional components, systems, subsystems, and elements other than those specifically shown and/or described herein. Additionally, or alternatively, the floss assembly partition 644 can omit one or more of the components, systems, subsystems, and elements that are specifically shown and/or described herein. In some embodiments, the floss assembly partition 644 can be positioned in a different manner than what is specifically illustrated in FIG. 6.

FIG. 7 is a side perspective view of another embodiment of the oral assembly 700. As shown in the embodiment illustrated in FIG. 7, the oral assembly 700 can include an assembly housing 702 including a charging port 746 and an oral device 704 including a first attachment 706 having a toothbrush head 748.

The charging port 746 can receive power from any suitable charging cable, including universal serial buses (“USBs”). The charging port 746 can be interconnected with various electronics and wiring in order to provide power to various device components (as described herein) of the oral assembly 700, such as the oral device 704, the first attachment 706, the oral device 704, the power button 110, the collection device 224, the first motor 334, and/or the second motor 336.

The charging port 746 can vary depending on the design requirements of the oral assembly 700, the assembly housing 702, the oral device 704, the first attachment 706, and/or any of the device components described herein. It is understood that the charging port 746 can include additional components, systems, subsystems, and elements other than those specifically shown and/or described herein. Additionally, or alternatively, the charging port 746 can omit one or more of the components, systems, subsystems, and elements that are specifically shown and/or described herein. In some embodiments, the charging port 746 is positioned in a different manner than what is specifically illustrated in FIG. 7.

The toothbrush head 748 can be coupled to the first attachment 706, as shown in FIG. 7. The toothbrush head 748 can vary depending on the design requirements of the oral assembly 700, the assembly housing 702, the oral device 704, the first attachment 706, and/or any of the device components described herein. It is understood that the toothbrush head 748 can include additional components, systems, subsystems, and elements other than those specifically shown and/or described herein. Additionally, or alternatively, the toothbrush head 748 can omit one or more of the components, systems, subsystems, and elements that are specifically shown and/or described herein. In some embodiments, the toothbrush head 748 is positioned in a different manner than what is specifically illustrated in FIG. 7.

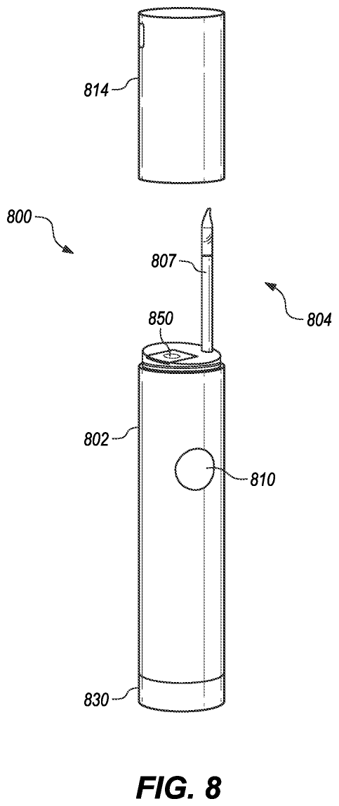

FIG. 8 is a perspective view of another embodiment of the oral assembly 800. As illustrated in the embodiment displayed in FIG. 8, the oral assembly 800 can include the assembly housing 802, the oral device 804, the second attachment 807, the power button 810, the oral device cover 814, the saliva storer 830, and a water reservoir cover 850. The oral device cover 814 is shown in the detached position, revealing the second attachment 807 and the water reservoir cover 850. As displayed in the embodiment of FIG. 8, the second attachment 807 is a water expulsion device that works in cooperation with the collection device 224.

The water reservoir cover 850 can be opened and closed so that water and/or other liquids can be added to the interior of the assembly housing 802. In other words, portions of the interior of the assembly housing 802 can be waterproofed and/or otherwise configured to retain water for use by the devices of the oral assembly 800.

The water reservoir cover 850 can vary depending on the design requirements of the oral assembly 800, the assembly housing 802, the oral device 804, the first attachment 706, the second attachment 807, and/or any of the device components described herein. It is understood that the water reservoir cover 850 can include additional components, systems, subsystems, and elements other than those specifically shown and/or described herein. Additionally, or alternatively, the water reservoir cover 850 can omit one or more of the components, systems, subsystems, and elements that are specifically shown and/or described herein. In some embodiments, the water reservoir cover 850 is positioned in a different manner than what is specifically illustrated in FIG. 8.

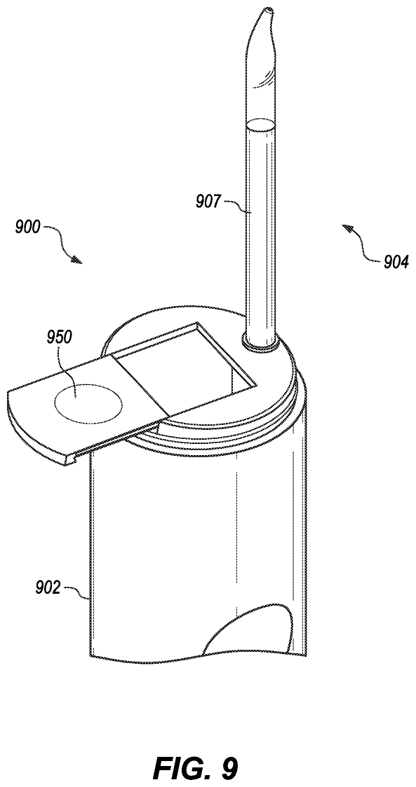

FIG. 9 is a top perspective view of one embodiment of a portion of the oral assembly 900. As illustrated in the embodiment displayed in FIG. 9, the oral assembly 900 can include the assembly housing 902, the oral device 904, the second attachment 907, and the water reservoir cover 950. In FIG. 9, the water reservoir cover 950 is shown in the open configuration, and a portion of the interior of the assembly housing 902 is viewable.

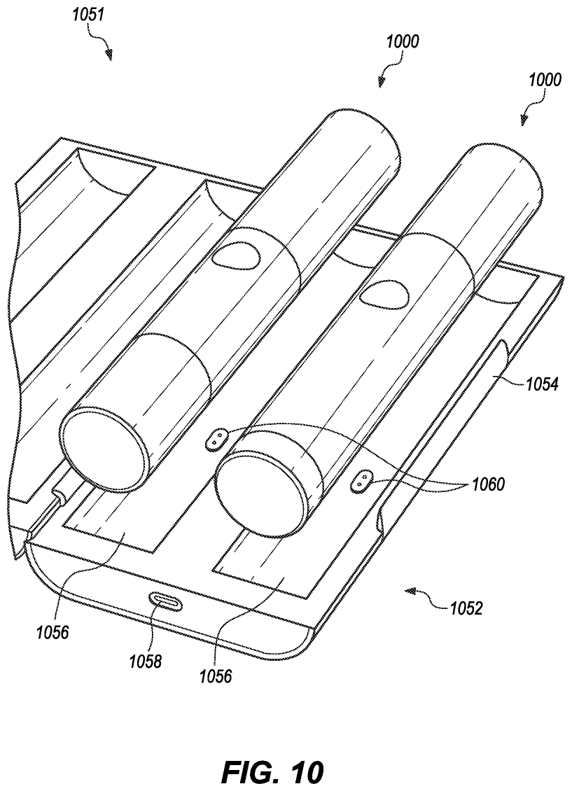

FIG. 10 is a perspective view of one embodiment of an oral system 1051, including a plurality of oral assemblies 1000 and a charger 1052. As shown in the embodiment illustrated in FIG. 10, the charger 1052 is shown in an open position with the plurality of oral assemblies 1000 in a detached and non-charging position.

The charger 1052 can charge and/or protect the oral assemblies 1000. The oral assemblies 1000 can be attached and in a charging position when placed in the interior portions of the charger 1052. The charger 1052 can be closed around the oral assemblies 1000 in order to protect the oral assemblies 1000.

The charger 1052 can vary depending on the design requirements of the oral assembly 1000 and/or the assembly housing 102 and/or any of the device and/or components described herein. It is understood that the charger 1052 can include additional components, systems, subsystems, and elements other than those specifically shown and/or described herein. Additionally, or alternatively, the charger 1052 can omit one or more of the components, systems, subsystems, and elements that are specifically shown and/or described herein. In some embodiments, the components of the charger 1052 can be positioned in a different manner than what is specifically illustrated in FIG. 10. In some embodiments, the charger 1052 can include a charger housing 1054, a charger chamber 1056, a charging port 1058, and a charger engager 1060.

The charger housing 1054 houses the various components of the charger 1052 and can include one or more chambers (such as the charger chamber 1056) for storing and/or charging the oral assemblies 1000.

The charger housing 1054 can vary depending on the design requirements of the oral assembly 1000, the assembly housing 102, the charger 1052, and/or any of the devices and/or components described herein. It is understood that the charger housing 1054 can include additional components, systems, subsystems, and elements other than those specifically shown and/or described herein. Additionally, or alternatively, the charger housing 1054 can omit one or more of the components, systems, subsystems, and elements that are specifically shown and/or described herein. In some embodiments, the components of the charger housing 1054 can be positioned in a different manner than what is specifically illustrated in FIG. 10.

The charger housing 1054 can include any number of suitable devices. The devices can be, or their functionality can include, integrated circuits, processors, controllers, microprocessors, diodes, capacitors, power storage elements, antennae, ASICs, sensors, image elements (e.g., CMOS, CCD imaging elements), amplifiers, ND, and D/A converters, associated differential amplifiers, buffers, microprocessors, optical collectors, transducer including electro-mechanical transducers, piezoelectric actuators, light-emitting electronics which include LEDs, logic, memory, clock, and transistors including active matrix switching transistors, and combinations thereof. The purpose and advantage of using standard ICs (in embodiments, CMOS, on single crystal silicon) are to have and use high quality, high performance, and high functioning circuit components that are also already commonly mass-produced with well-known processes, and which provide a range of functionality and generation of data far superior to that produced by a passive means.

Components within electronic devices or devices are described herein and include those components described above. A component can be one or more of any of the electronic devices described above and/or may include a photodiode, LED, TUFT, electrode, semiconductor, other light-collecting/detecting components, transistor, contact pad capable of contacting a device component, thin-film devices, circuit elements, control elements, microprocessors, interconnects, contact pads, capacitors, resistors, inductors, a memory element, power storage element, antenna, logic element, buffer and/or other passive or active components. A device component may be connected to one or more contact pads as known in the art, such as metal evaporation, wire bonding, application of solids or conductive pastes, and the like. In some embodiments, the charger chamber 1056, the charging port 1058, and the charger engager 1060 are integrally formed with the charger housing 1054.

The charger chamber 1056 can be configured to receive the oral assemblies 1000 and can be integrally formed within the charger 1052. The charger chambers 1056 can also be configured to include charging elements (such as the charger engager 1060) that charge the oral assemblies 1000 while the oral assemblies 1000 are seated within the charger chambers 1056. It is understood that any number of charger chambers 1056 can be implemented within the charger 1052 so that any number of oral assemblies 1000 can be protected and/or charged.

The charger chamber 1056 can vary depending on the design requirements of the oral assembly 1000, the assembly housing 102, the charger 1052, the charger housing 1054, and/or any of the devices and/or components described herein. It is understood that the charger chamber 1056 can include additional components, systems, subsystems, and elements other than those specifically shown and/or described herein. Additionally, or alternatively, the charger chamber 1056 can omit one or more of the components, systems, subsystems, and elements that are specifically shown and/or described herein. In some embodiments, the components of the charger chamber 1056 can be positioned in a different manner than what is specifically illustrated in FIG. 10.

The charging port 1058 can receive power from any suitable charging cable, including universal serial buses (“USBs”). The charging port 1058 can be substantially similar to the charging port 746. The charging port 1058 can be interconnected with various electronics and wiring in order to provide power to various device components (as described herein) of the charger 1052, such as the charger housing 1054, the charger chamber 1056, and/or the charger engager 1060.

The charging port 1058 can vary depending on the design requirements of the charger 1052, such as the charger housing 1054, the charger chamber 1056, the charger engager 1060, and/or any of the devices and/or components described herein. It is understood that the charging port 1058 can include additional components, systems, subsystems, and elements other than those specifically shown and/or described herein. Additionally, or alternatively, the charging port 1058 can omit one or more of the components, systems, subsystems, and elements that are specifically shown and/or described herein. In some embodiments, the charging port 1058 is positioned in a different manner than what is specifically illustrated in FIG. 10.

The charger engager 1060 can provide power to the oral assembly 1000. The charger engager 1060 can vary depending on the design requirements of the oral assembly 1000, the charger 1052, the charger housing 1054, the charger chamber 1056, the charging port 1058, and/or any of the devices and/or components described herein. It is understood that the charger engager 1060 can include additional components, systems, subsystems, and elements other than those specifically shown and/or described herein. Additionally, or alternatively, the charger engager 1060 can omit one or more of the components, systems, subsystems, and elements that are specifically shown and/or described herein. In some embodiments, the charger engager 1060 is positioned in a different manner than what is specifically illustrated in FIG. 10.

The charger engager 1060 can include spring-loaded pogo pins. While the oral assembly 1000 is in contact with the charger engager 1060, the charger engager 1060 is in electrical contact with a conductive portion (not shown) of the oral assembly 1000 so that the oral assembly 1000 can receive power from the charger engager 1060.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications can be made while remaining within the spirit and scope herein.

It is understood that although a number of different embodiments of the systems and methods have been illustrated and described herein, one or more features of any one embodiment can be combined with one or more features of one or more of the other embodiments, provided that such combination satisfies the intent of the present invention.

While a number of exemplary aspects and embodiments of the user interface and methods have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. An oral assembly for use within a mouth of a user, the oral assembly comprising:
an oral device that is configured to be usable within the mouth of the user to provide oral health care treatment; and
a collection device that is coupled to the oral device, the collection device being movable between (i) a collection mode wherein the collection device collects saliva from the mouth of the user, and (ii) an expulsion mode wherein the collection device expels a liquid into the mouth of the user.

2. The oral assembly of claim 1 wherein in the expulsion mode, the collection device cooperates with the oral device to floss teeth within the mouth of the user with the liquid.

3. The oral assembly of claim 1 further comprising a motor that is coupled to the collection device, the motor being configured to move the collection device between the collection mode and the expulsion mode.

4. The oral assembly of claim 1 further comprising an assembly housing that houses the collection device.

5. The oral assembly of claim 4 further comprising a housing cap that is removably attachable to the assembly housing, the assembly housing having an assembly housing axis, the housing cap being configured to be rotatable about the assembly housing axis.

6. The oral assembly of claim 5 wherein the assembly housing includes a first attachment aperture that is configured to receive a first attachment, the first attachment aperture being configured to activate the first attachment upon insertion.

7. The oral assembly of claim 6 wherein the assembly housing includes a second attachment aperture that is configured to receive a second attachment, the second attachment aperture being configured to activate the second attachment upon insertion.

8. The oral assembly of claim 7 wherein the housing cap includes a housing cap aperture, and rotation of the housing cap exposes the first attachment aperture and the second attachment aperture when they are aligned with the housing cap aperture.

9. The oral assembly of claim 7 wherein the housing cap is selectively lockable when at least one of the first attachment aperture and the second attachment aperture is exposed.

10. The oral assembly of claim 1 further comprising a collection tube that is coupled to the collection device, the collection tube being configured to receive saliva that is collected by the collection device from the mouth of the user.

11. The oral assembly of claim 1 further comprising a saliva storer that is configured to store the saliva collected by the collection device from the mouth of the user.

12. An oral assembly for use within a mouth of a user, the oral assembly comprising:
an oral device that is configured to be usable within the mouth of the user to provide oral health care treatment;
a collection device that is coupled to the oral device, the collection device being movable between (i) a collection mode wherein the collection device collects saliva from the mouth of the user, and (ii) an expulsion mode wherein the collection device expels a liquid into the mouth of the user;
a collection tube that is coupled to the collection device, the collection tube being configured to receive saliva that is collected by the collection device from the mouth of the user; and
a saliva storer that is configured to store the saliva collected by the collection device from the mouth of the user.

13. The oral assembly of claim 12 further comprising an extension tube that selectively connects to at least one of the collection tube and the saliva storer.

14. The oral assembly of claim 13 wherein the extension tube includes an extension tube cap that threadedly engages the extension tube to secure the contents of the saliva storer.

15. The oral assembly of claim 12 wherein the saliva storer is selectively detachable from the collection device.

16. The oral assembly of claim 12 wherein the saliva storer includes a storer disc having a storer disc tube that is selectively sealable using a sealing cap.

17. The oral assembly of claim 16 wherein the storer disc is configured to be removable from the saliva storer.

18. The oral assembly of claim 12 further comprising a device cover that is configured to (i) cover the collection device and (ii) selectively attach to the oral assembly.

19. The oral assembly of claim 12 wherein in the expulsion mode, the collection device cooperates with the oral device to floss teeth within the mouth of the user with the liquid.

20. An oral assembly for use within a mouth of a user, the oral assembly comprising:
an oral device that is configured to be usable within the mouth of the user to provide oral health care treatment;
a collection device that is coupled to the oral device, the collection device being movable between (i) a collection mode wherein the collection device collects saliva from the mouth of the user, and (ii) an expulsion mode wherein the collection device expels a liquid into the mouth of the user;
a collection tube that is coupled to the collection device, the collection tube being configured to receive saliva that is collected by the collection device from the mouth of the user;
a motor coupled to the collection device, the motor being configured to move the collection device between the collection mode and the expulsion mode; and
an assembly housing that houses the collection device, the assembly housing including a first attachment aperture that is configured to receive a first attachment, the first attachment being activated by inserting the first attachment into the first attachment aperture, the assembly housing including a second attachment aperture that is configured to receive a second attachment, the second attachment being activated by inserting the second attachment into the second attachment aperture.
